SEQUENCE LISTING (1 sequence)

SEQ ID NO: 1 - RNA, 20 nt, Artificial Sequence
  Synthetic polynucleotide
ggccgacuacuacgccaagg

(19) United States
(12) Patent Application Publication
(10) Pub. No.: US 2024/0197782 A1
(43) Pub. Date: Jun. 20, 2024

(54) METHODS AND COMPOSITIONS FOR GENETIC MODIFICATION AND THERAPEUTIC USE OF IMMUNE CELLS

(71) Applicant: BOARD OF REGENTS, THE UNIVERSITY OF TEXAS SYSTEM, Austin, TX (US)

(72) Inventors: Cassian YEE, Houston, TX (US); Yongming XUE, Houston, TX (US); Farah HASAN, Houston, TX (US)

(73) Assignee: BOARD OF REGENTS, THE UNIVERSITY OF TEXAS SYSTEM, Austin, TX (US)

(21) Appl. No.: 18/555,846

(22) PCT Filed: Apr. 20, 2022
(86) PCT No.: PCT/US2022/025612

§ 371 Date: Oct. 17, 2023

Related U.S. Application Data
(60) Provisional application No. 63/177,053, filed on Apr. 20, 2021.

(51) Int. Cl. A61K 35/17; A61P 35/00; C12N 5/0783; C12N 9/22; C12N 15/11

(52) U.S. Cl. CPC A61K 35/17; A61P 35/00; C12N 5/0636; C12N 9/22; C12N 15/111; C12N 2310/20; C12N 2510/00

(57) ABSTRACT

Aspects of the present disclosure relate to methods and compositions for genetic modification of immune cells to generate immune cells that do not secrete TGF-β1. Also disclosed are methods for use of such genetically modified immune cells, including immunotherapeutic methods for the treatment of cancer. Further aspects of the disclosure are directed to human CD8⁺ cells comprising a genetic modification that prevents the cells from secreting TGF-β1.

This application claims priority of U.S. Provisional Patent Application No. 63/177,053, filed Apr. 20, 2021, which is hereby incorporated by reference in its entirety.

### SEQUENCE LISTING

The instant application contains a Sequence Listing which has been submitted in ASCII format and is hereby incorporated by reference in its entirety. Said ASCII copy, created on Apr. 19, 2022, is named MDAC_P1296WO_Sequence_Listing.txt and is 558 bytes in size.

### BACKGROUND

### I. Field of the Invention

Aspects of this invention relate to at least the fields of immunology, cancer biology, and medicine.

### II. Background

Adoptive T cell therapy (ACT), where tumor-reactive T lymphocytes are isolated from peripheral blood or tumor tissue, genetically engineered, expanded in vitro and infused into patients, has been shown to induce clinical responses in patients with leukemia, lymphoma, metastatic melanoma, and other malignancies. The effector functions of cytotoxic T lymphocytes (CTLs) are suppressed by multiple factors, including TGF-β1. In an attempt to overcome the suppressive role of TGF-β1 on the effector functions of CTLs, T cells have been engineered to express the dominant negative form of TGF-receptor (TGFBRII-DNR) or knock out TGF-β receptor to block TGF-β1 perception. These approaches seek to maintain the anti-tumor efficacy of CTLs in high-TGF-1 microenvironment. However, CD8⁺ T cells require the perception of TGF-β1 to form resident memory T cells (T_{RM}), which may be crucial to the long-term anti-tumor effect of ACT. Current strategies abrogate CD8⁺ T cells' capability to form T_{RM} and dampen ACT efficacies. Therefore, there remains a need for superior ACT compositions and methods with enhanced efficacy, including therapeutic CD8+ T cells that retain the ability to form T_{RM}.

### SUMMARY

Aspects of the present disclosure address certain needs in the art by providing immune cells (e.g., T cells such as CD8⁺ T cells) that do not secrete TGF-β1 (e.g., TGFB1 knockout cells). Such cells, as disclosed herein, may be effective for targeting and killing of tumor cells, and may retain the ability to form resident memory T cells (T_{RM}). Also disclosed are compositions comprising such cells, methods for generation of such cells, and methods for use of such cells for the treatment of cancer.

Embodiments of the disclosure include immune cells (e.g., T cells such as CD8⁺ T cells, CD4⁺ T cells, natural killer cells, CAR-T cells, and TCR-T cells), tumor-infiltrating lymphocytes, genetically modified cells, nucleic acids (e.g., RNA such as gRNA), vectors, enzymes (e.g., Cas nucleases such as Cas9 nuclease), pharmaceutical compositions, methods for treating a subject having cancer, methods for genetically modifying a cell, and methods for generating a TGFB1 mutation. Cells of the present disclosure (e.g., immune cells, tumor-infiltrating lymphocytes, cytotoxic T cells, etc.) can include at least 1, 2, 3, or more of the following: a genetic modification of TGFB1, a frameshift mutation of TGFB1, a nonsense mutation of TGFB1, a deletion of TGFB1, a modification of a region of exon 1 of TGFB1, an epigenetic modification of TGFB1, and a genetic modification of a regulatory region (e.g., promoter, enhancer, etc.) that controls expression of TGFB1. Methods of the present disclosure can include at least 1, 2, 3, 4, 5 of more of the following steps: obtaining an immune cell from a subject, obtaining a tumor infiltrating lymphocyte from a subject, obtaining a CD8⁺ T cell from a subject, expanding a cell obtained from a subject, culturing a cell obtained from a subject, genetically modifying a human CD8⁺ T cell, modifying an immune cell to generate a genetically modified immune cell that does not secrete TGF-β1, introducing a Cas nuclease and a guide RNA targeted to TGFB1 into a human CD8⁺ T cell, administering a genetically modified immune cell to a subject, administering to a subject a human CD8⁺ T cell that does not secrete TGF-β1, diagnosing a subject for cancer, administering a cancer therapy to a subject, and determining a subject to be resistant to a cancer therapy. Compositions of the disclosure may comprise at least 1, 2, 3, 4, or more of the following components: an immune cell, a human CD8⁺ T cell, a tumor-infiltrating lymphocyte, a genetically modified immune cell, a Cas nuclease, a guide RNA, a vector, a plasmid, a transfection reagent, an excipient, and a cancer therapeutic. It is specifically contemplated that one of more of the recited steps and/or components may be excluded from certain embodiments of the disclosure.

In some aspects, disclosed herein is a method for treating a subject having cancer, the method comprising administering to the subject a human CD8⁺ T cell that does not secrete TGF-β1. In some embodiments, the human CD8⁺ T cell does not express TGF-β1. In some embodiments, the human CD8⁺ T cell expresses a mutant TGF-β1 protein. In some embodiments, the human CD8⁺ T cell was derived from a CD8⁺ T cell from the subject. In some embodiments, the human CD8⁺ T cell from the subject is a tumor-infiltrating lymphocyte. In some embodiments, the human CD8⁺ T cell comprises a mutation of TGFB1. In some embodiments, the mutation is a frameshift mutation. In some embodiments, the mutation is a nonsense mutation. In some embodiments, the mutation comprises a modification of (e.g., a mutation in) a region of exon 1 of TGFB1. In some embodiments, the region of exon 1 comprises the sequence of SEQ ID NO: 1. In some embodiments, the cancer is melanoma. In some embodiments, the cancer is recurrent cancer. In some embodiments, the subject was previously treated for cancer with a previous treatment. In some embodiments, the subject was determined to be resistant to the previous treatment. In some embodiments, the method comprises administering to the subject a population of human CD8⁺ T cells comprising the human CD8⁺ T cell. In some embodiments, each cell of the population of human CD8⁺ T cells does not secrete TGF-β1. In some embodiments, the method further comprises administering an additional cancer therapy. In some embodiments, the additional cancer therapy is a chemotherapy, radiotherapy, or immunotherapy.

Also disclosed is a population of CD8⁺ T cells comprising the human CD8⁺ T cell. In some embodiments, each cell of the population of CD8⁺ T cells does not secrete TGF-1. In some embodiments, a portion of the population of CD8⁺ T cells does not secrete TGF-β1. In some embodiments, TGF-β1 secretion is undetectable in the population of CD8+ T cells. In some embodiments, TGF-β1 secretion is substantially decreased in the population of CD8⁺ T cells compared with a population of control cells. In some embodiments, TGF-β1 secretion is decreased in the population of CD8⁺ T cells by at least, at most, or about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99%, 99.1%, 99.2%, 99.3%, 99.4%, 99.5%, 99.6%, 99.7%, 99.8%, 99.9%, or any range or value derivable therein, compared with a population of control cells. In some embodiments, a portion of the population of CD8⁺ T cells does not express TGF-β1. In some embodiments, TGF-β1 expression is undetectable in the population of CD8+ T cells. In some embodiments, TGF-β1 expression is substantially decreased in the population of CD8⁺ T cells compared with a population of control cells (e.g., wild-type cells, untreated cells, cells treated with control gene editing reagents, etc.). In some embodiments, TGF-β1 expression is decreased by at least, at most, or about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99%, 99.1%, 99.2%, 99.3%, 99.4%, 99.5%, 99.6%, 99.7%, 99.8%, 99.9%, or any range or value derivable therein. In some embodiments, TGF-β1 expression is decreased by at least 80%, 90%, 95%, or 99%. In some embodiments, TGF-β1 expression is decreased by at least 90%.

Disclosed herein, in some embodiments, is a method for genetically modifying a human CD8⁺ T cell, the method comprising introducing into the cell a Cas nuclease and a guide RNA targeted to TGFB1. In some embodiments, the guide RNA is targeted to a region of exon 1 of TGFB1. In some embodiments, the region of exon 1 comprises the sequence of SEQ ID NO:1 (or a portion thereof). In some embodiments, the Cas nuclease is Cas9. In some embodiments, the Cas nuclease is Cas12a. In some embodiments, a mutation is generated in TGFB1 in the cell. In some embodiments, the mutation is generated using the Cas nuclease. In some embodiments, the mutation is a frameshift mutation. In some embodiments, the mutation is a nonsense mutation. In some embodiments, the Cas nuclease and the guide RNA are introduced into the cell via transfection. In some embodiments, the Cas nuclease and the guide RNA are introduced into the cell via electroporation. In some embodiments, the method further comprises obtaining the human CD8⁺ T cell from a subject. In some embodiments, the human CD8⁺ T cell is a tumor-infiltrating lymphocyte.

Also disclosed herein, in some embodiments, is a method for treating a subject having cancer, the method comprising (a) modifying immune cells from the subject to generate genetically modified immune cells that do not secrete TGF-β1; and (b) administering the genetically modified immune cells to the subject. In some embodiments, the immune cells do not express TGF-β1. In some embodiments, the immune cells express a mutant TGF-β1 protein. In some embodiments, the immune cells are natural killer T cells. In some embodiments, the immune cells are modified to express a cloned T cell receptor (TCR) or a chimeric antigen receptor (CAR). In some embodiments, the immune cells are engineered CAR-T cells or engineered TCR-T cells. In some embodiments, the immune cells are T cells. In some embodiments, the T cells are CD4+ T cells. In some embodiments, the T cells are CD8+ T cells. In some embodiments, the immune cells are tumor-infiltrating lymphocytes. In some embodiments, modifying the immune cells comprises generating a mutation in TGFB1 in the immune cells. In some embodiments, the mutation is a frameshift mutation. In some embodiments, the mutation is a nonsense mutation. In some embodiments, the mutation is a mutation in exon 1 of TGFB1. In some embodiments, modifying the immune cells comprises targeting a region of exon 1 of TGFB1 using a Cas nuclease and a guide RNA. In some embodiments, the region of exon 1 comprises the sequence of SEQ ID NO:1. In some embodiments, the subject is a human subject.

Further disclosed, in certain aspects, is a human immune cell comprising a genetic modification that prevents the cell from secreting TGF-β1. In some embodiments, the immune cell is a T cell. In some embodiments, the T cell is a CD8⁺ T cell. In some embodiments, the T cell is a CD4⁺ T cell. In some embodiments, the immune cell is a natural killer T cell. In some embodiments, the immune cell expresses a cloned TCR or a CAR. In some embodiments, the immune cell is an engineered CAR-T cell or an engineered TCR-T cell. In some embodiments, the genetic modification is a frameshift mutation of TGFB1. In some embodiments, the genetic modification is a nonsense mutation of TGFB1. In some embodiments, the genetic modification is a deletion of TGFB1. In some embodiments, the genetic modification comprises a modification of (e.g., a mutation in) a region of exon 1 of TGFB1. In some embodiments, the region of exon 1 comprises the sequence of SEQ ID NO:1. In some embodiments, the human immune cell is or was derived from a tumor-infiltrating lymphocyte. Also disclosed is a pharmaceutical composition comprising a cell of the present disclosure and, in some cases, a pharmaceutically acceptable excipient. Also disclosed is a population of human immune cells comprising the human immune cell. In some embodiments, secretion of TGF-β1 is undetectable in the population of human immune cells. In some embodiments, secretion of TGF-β1 is decreased by at least, at most, or about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99%, 99.1%, 99.2%, 99.3%, 99.4%, 99.5%, 99.6%, 99.7%, 99.8%, 99.9%, or any range or value derivable therein, compared with a population of control cells (e.g., a population of immune cells that have not been genetically modified).

Throughout this application, the term “about” is used to indicate that a value includes the inherent variation of error for the measurement or quantitation method.

The use of the word “a” or “an” when used in conjunction with the term “comprising” may mean “one.” but it is also consistent with the meaning of “one or more.” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise.” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments described herein in the context of the term “comprising” may also be implemented in the context of the term “consisting of” or “consisting essentially of.”

“Individual, “subject.” and “patient” are used interchangeably and can refer to a human or non-human.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Any step in a method described herein can apply to any other method. Moreover, any method described herein may have an exclusion of any step or combination of steps. The embodiments in the Example section are understood to be embodiments that are applicable to all aspects of the technology described herein.

Use of the one or more sequences or compositions may be employed based on any of the methods described herein. Other embodiments are discussed throughout this application. Any embodiment discussed with respect to one aspect of the disclosure applies to other aspects of the disclosure as well and vice versa.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1D show data demonstrating that CRISPR/Cas9 RNP efficiently depletes TGFB1 gene in CD8⁺ T cells. FIG. 1A shows the sequence (SEQ ID NO:1), location, Indel percentage and knockout score of the TGFB1 gRNA. FIG. 1B shows Sanger sequencing results of TGFB1 gene fragment flanking the target sequence of the gRNA in the non-target (NT) control and TGFB1 knockout CD8⁺ T cells. FIG. 1C shows representative plots of membrane TGF-β1 (LAP) and CD8 levels of Fluorescence Minus One (FMO) control, NT control and TGFB1 knockout CD8⁺ T cells on Day 2 after stimulation with α-CD3/α-CD28 beads. FIG. 1D shows levels of total secreted TGF-β1 of NT control and TGFB1 knockout CD8⁺ T cells on Day 2 after stimulation with α-CD3/α-CD28 beads.

FIGS. 2A-2D show data demonstrating that TGFB1 knockout in poly-clonally activated CD8⁺ T cells enhances cytokines production and does not affect T_{RM} formation. FIG. 2A shows representative plots of intracellular TNF-α, IFN-γ. Granzyme B and IL-2 levels of control and TGFB1-depleted CD8⁺ T cells after re-stimulation. FIG. 2B shows percentages of IFN-γ⁺, TNF-α⁺, IFN-γ⁺ TNF-α⁺ and IL-2⁺ CD8⁺ T cells after re-stimulation.

FIG. 2C shows mean fluorescence intensity (MFI) levels of intracellular Granzyme B after re-stimulation. Results between experimental groups were compared using Student's t test. n=3. Statistical significance is displayed as N.S., p >=0.05. * p<0.05. ** p<0.01. FIG. 2D shows representative plots of CD103 and CD69 levels of control and TGFB1-depleted CD8⁺ T cells after cultured with exogenous TGF-β1 under hypoxic conditions. Error bars represent standard error of the mean (SEM).

FIG. 3 shows flow cytometry data of NT control and TGFB1-depleted TCR-T cells after rapid expansion protocol (REP).

FIGS. 4A-4C show data demonstrating that TGFB1 knockout enhances the production of cytokines of TCR-T cells. FIG. 4A shows representative plots of intracellular TNF-α, IFN-γ, Granzyme B and IL-2 levels of control and TGFB1 knockout TCR-T cells after co-culture with Mel526 cell (E:T ratio=5:1) for 16 hr. FIG. 4B shows percentages of IFN-γ+, TNF-α+, IFN-γ+ TNF-α+ and IL-2+ TCR-T cells. FIG. 4C shows mean fluorescence intensity (MFI) levels of intracellular Granzyme B. Results between experimental groups were compared using Student's t test. n=3. Statistical significance is displayed as N.S., p >=0.05, * p<0.05. ** p<0.01. Error bars represent SEM.

FIGS. 5A-5C shows data demonstrating that TGFB1 knockout enhances the cytolytic activity of TCR-T cells. FIG. 5A shows cytotoxicity of NT control and TGFB1 knockout TCR-T cells co-cultured with Mel526 cells at various E:T ratios, measured by chromium release assay. FIG. 5B cytotoxicity of NT control and TGFB1 knockout TCR-T cells co-cultured with Mel526 cells at various E:T ratios. TCR-T cells were treated with 10 ng/ml TGF-β1 for 4 days before chromium release assay. FIG. 5C shows the combination of the data shows in FIG. 5A and FIG. 5B. Results between experimental groups were compared using Student's t test. n=4. Statistical significance is displayed as N.S., p >=0.05. * p<0.05, ** p<0.01. Error bars represent SEM.

### DETAILED DESCRIPTION OF THE INVENTION

The present disclosure is based, at least in part, on the development of immune cells (including T cells such as CD8⁺ T cells) having a genetic modification that reduces or eliminates expression of TGF-β1 in the cells, and further on the discovery that such modified cells have enhanced anti-tumor efficacy and retain the ability to form resident memory T cells (T_{RM}). Certain aspects of the disclosure are directed to genetically modified immune cells (e.g., human CD8⁺ T cells) having a knockout mutation in TGFB1, and to methods of use of such cells for treatment of cancer. Also disclosed are methods for genetic modification of TGFB1 in immune cells using genetic editing techniques, including CRISPR/Cas-based gene editing.

### I. Gene Editing Systems

Certain aspects of the present disclosure relate to methods and compositions for gene editing (also “genetic engineering”), useful in the generation of one or more genetic modifications in a cell. As used herein, a “genetic modification,” describes a region of a genome of a cell that has been altered from its native (i.e., endogenous) sequence. A genetic modification may be developed via artificial editing of a gene or other genetic material. In some embodiments, a genetic modification is a mutation of a gene. Various types of gene mutations are recognized in the art and contemplated herein. In some embodiments, a mutation is an insertion, a deletion, a point mutation, a frameshift mutation, or a nonsense mutation. One or more of these mutations may be excluded from embodiments of the disclosure. In some embodiments, a mutation prevents expression of a gene (i.e., is a knockout mutation). In some embodiments, a mutation causes production of a mutant form of a protein. In some embodiments, a genetic modification of the disclosure is a mutation of TGFB1. In some embodiments, a mutation of TGFB1 is a mutation in a region of exon 1, 2, 3, 4, 5, 6, or 7 of TGFB1. In some embodiments, the mutation is a mutation in a region of exon 1 of TGFB1. In some embodiments, a genetic modification of the disclosure is a mutation of TGFB2. In some embodiments, a mutation of TGFB2 is a mutation in a region of exon 1, 2, 3, 4, 5, 6, or 7 of TGFB2. In some embodiments, the mutation is a mutation in a region of exon 1 of TGFB2. In some embodiments, a genetic modification of the disclosure is a mutation of TGFB3. In some embodiments, a mutation of TGFB3 is a mutation in a region of exon 1, 2, 3, 4, 5, 6, or 7 of TGFB3. In some embodiments, the mutation is a mutation in a region of exon 1 of TGFB3.

Certain embodiments of the disclosure are directed to the use of gene editing techniques to generate a knockout mutation in a gene in a population of cells. The disclosed techniques may eliminate expression of the gene in some or all of the cells in the population. In some embodiments, expression of the gene is not detectable in the population of cells (e.g., measured by mRNA and/or protein expression). In some embodiments, expression of the gene is substantially decreased in the population of cells compared with cells not having the knockout mutation. In some embodiments, expression of the gene (e.g., measured by mRNA and/or protein expression) is decreased by at least, at most, or about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99%, 99.1%, 99.2%, 99.3%, 99.4%, 99.5%, 99.6%, 99.7%, 99.8%, 99.9%, or any range or value derivable therein. In some embodiments, expression of the gene is decreased by at least 80%, 90%, 95%, or 99%. In some embodiments, expression of the gene is decreased by at least 90%.

Various methods and systems for gene editing are known in the art and include, for example, zinc finger nuclease (ZFN)-based gene editing, transcription activator-like effector nuclease (TALEN)-based gene editing, and CRISPR/Cas-based gene editing. In some embodiments, methods of the present disclosure comprise CRISPR/Cas-based gene editing, which comprises the use of components of a CRISPR system, for example a guide RNA (gRNA) and a Cas nuclease.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. A Cas nuclease and a gRNA can be introduced into the cell indirectly via introduction of one or more nucleic acids (e.g., vectors) encoding for the Cas nuclease and/or the gRNA. A Cas nuclease and a gRNA can be introduced into the cell directly by introduction of a Cas nuclease protein and a gRNA molecule. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA may be targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% sequence complementarity along the length of the tracr mate sequence when optimally aligned.

One or more vectors driving expression of one or more elements of a CRISPR system can be introduced into a cell such that expression of the elements of the CRISPR system direct formation of a CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding a Cas protein (also “Cas nuclease”). Non-limiting examples of Cas proteins include Cas1, Cas1B, Cas2, Cas3, Cas4, Cas5, Cas6, Cas7, Cas8, Cas9 (also known as Csn1 and Csx12), Cas10, Cas12a (Cpf1), Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The Cas nuclease can be Cas9 (e.g., from S. pyogenes or S. pneumonia). The Cas nuclease can be Cas12a. The Cas nuclease can direct cleavage of one or both strands at the location of a target sequence, such as within the target sequence and/or within the complement of the target sequence. The vector can encode a Cas nuclease that is mutated with respect to a corresponding wild-type enzyme such that the mutated Cas nuclease lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is or is more than 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The Cas nuclease may be part of a fusion protein comprising one or more heterologous protein domains. A Cas nuclease fusion protein may comprise any additional protein sequence, and optionally a linker sequence between any two domains. Examples of protein domains that may be fused to a Cas nuclease, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A Cas nuclease may be fused to a gene sequence encoding a protein or a fragment of a protein that bind DNA molecules or bind other cellular molecules, including but not limited to maltose binding protein (MBP), S-tag. Lex A DNA binding domain (DBD) fusions, GAL4A DNA binding domain fusions, and herpes simplex virus (HSV) BP16 protein fusions. Additional domains that may form part of a fusion protein comprising a Cas nuclease are described in US 20110059502, incorporated herein by reference.

### II. Therapeutic Methods

Aspects of the disclosure are directed to compositions and methods for therapeutic use. The compositions of the disclosure may be used for in vivo, in vitro, or ex vivo administration. The route of administration of the composition may be, for example, intratumoral, intravenous, intramuscular, intraperitoneal, subcutaneous, intraarticular, intrasynovial, intrathecal, oral, topical, through inhalation, or through a combination of two or more routes of administration.

### A. Cancer Therapy

In some embodiments, the disclosed methods comprise administering a cancer therapy to a subject or patient. The cancer therapy may be chosen based on the expression level measurements, alone or in combination with the clinical risk score calculated for the subject. In some embodiments, the cancer therapy comprises a local cancer therapy. In some embodiments, the cancer therapy excludes a systemic cancer therapy. In some embodiments, the cancer therapy excludes a local therapy. In some embodiments, the cancer therapy comprises a local cancer therapy without the administration of a system cancer therapy. In some embodiments, the cancer therapy comprises an immunotherapy, which may be a checkpoint inhibitor therapy or an adoptive cell therapy. In some embodiments, the cancer therapy is an adoptive T cell therapy (ACT). Any of these cancer therapies may also be excluded. Combinations of these therapies may also be administered.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In some embodiments, the cancer is melanoma.

In some embodiments, the cancer is a recurrent cancer. In some embodiments, the cancer is Stage I cancer. In some embodiments, the cancer is Stage II cancer. In some embodiments, the cancer is Stage III cancer. In some embodiments, the cancer is Stage IV cancer.

It is contemplated that a cancer treatment may exclude any of the cancer treatments described herein. Furthermore, embodiments of the disclosure include patients that have been previously treated with a therapy described herein, are currently being treated with a therapy described herein, or have not been treated with a therapy described herein. In some embodiments, the patient is one that has been determined to be resistant to a therapy described herein. In some embodiments, the patient is one that has been determined to be sensitive to a therapy described herein.

### B. Cancer Immunotherapy

In some embodiments, the methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Various immunotherapies are known in the art, and examples are described below.

### 1. Checkpoint Inhibitors and Combination Treatment

Embodiments of the disclosure may include administration of immune checkpoint inhibitors, examples of which are further described below. As disclosed herein, “checkpoint inhibitor therapy” (also “immune checkpoint blockade therapy”, “immune checkpoint therapy”, “ICT.” “checkpoint blockade immunotherapy.” or “CBI”), refers to cancer therapy comprising providing one or more immune checkpoint inhibitors to a subject suffering from or suspected of having cancer.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC. Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO2001/014424, WO 98/42752; WO 20000/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (sec, e.g., WO2001/014424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

c. LAG3

Another immune checkpoint that can be targeted in the methods provided herein is the lymphocyte-activation gene 3 (LAG3), also known as CD223 and lymphocyte activating 3. The complete mRNA sequence of human LAG3 has the Genbank accession number NM_002286. LAG3 is a member of the immunoglobulin superfamily that is found on the surface of activated T cells, natural killer cells, B cells, and plasmacytoid dendritic cells. LAG3's main ligand is MHC class II, and it negatively regulates cellular proliferation, activation, and homeostasis of T cells, in a similar fashion to CTLA-4 and PD-1, and has been reported to play a role in Treg suppressive function. LAG3 also helps maintain CD8⁺ T cells in a tolerogenic state and, working with PD-1, helps maintain CD8 exhaustion during chronic viral infection. LAG3 is also known to be involved in the maturation and activation of dendritic cells. Inhibitors of the disclosure may block one or more functions of LAG3 activity.

In some embodiments, the immune checkpoint inhibitor is an anti-LAG3 antibody (e.g., a human antibody, a humanized antibody, or a chimeric antibody), an antigen binding fragment thereof, an immunoadhesin, a fusion protein, or oligopeptide.

Anti-human-LAG3 antibodies (or VH and/or VL domains derived therefrom) suitable for use in the present methods can be generated using methods well known in the art. Alternatively, art recognized anti-LAG3 antibodies can be used. For example, the anti-LAG3 antibodies can include: GSK2837781, IMP321, FS-118, Sym022, TSR-033, MGD013, BI754111, AVA-017, or GSK2831781. The anti-LAG3 antibodies disclosed in: U.S. Pat. No. 9,505,839 (BMS-986016, also known as relatlimab); U.S. Pat. No. 10,711,060 (IMP-701, also known as LAG525); U.S. Pat. No. 9,244,059 (IMP731, also known as H5L7BW); U.S. Pat. No. 10,344,089 (25F7, also known as LAG3.1); WO 2016/028672 (MK-4280, also known as 28G-10); WO 2017/019894 (BAP050); Burova E., et al., J. ImmunoTherapy Cancer, 2016; 4(Supp. 1):P195 (REGN3767); Yu, X., ct al., mAbs, 2019; 11:6 (LBL-007) can be used in the methods disclosed herein. These and other anti-LAG-3 antibodies useful in the claimed invention can be found in, for example: WO 2016/028672, WO 2017/106129, WO 2017062888, WO 2009/044273, WO 2018/069500, WO 2016/126858, WO 2014/179664, WO 2016/200782, WO 2015/200119, WO 2017/019846, WO 2017/198741, WO 2017/220555, WO 2017/220569, WO 2018/071500, WO 2017/015560; WO 2017/025498, WO 2017/087589, WO 2017/087901, WO 2018/083087, WO 2017/149143, WO 2017/219995, US 2017/0260271, WO 2017/086367, WO 2017/086419, WO 2018/034227, and WO 2014/140180. The teachings of cach of the aforementioned publications are hereby incorporated by reference. Antibodies that compete with any of these art-recognized antibodies for binding to LAG3 also can be used.

In some embodiments, the inhibitor comprises the heavy and light chain CDRs or VRs of an anti-LAG3 antibody. Accordingly, in one embodiment, the inhibitor comprises the CDR1. CDR2, and CDR3 domains of the VH region of an anti-LAG3 antibody, and the CDR1, CDR2 and CDR3 domains of the VL region of an anti-LAG3 antibody. In another embodiment, the antibody has at least about 70, 75, 80, 85, 90, 95, 97, or 99% (or any derivable range therein) variable region amino acid sequence identity with the above-mentioned antibodies.

d. TIM-3

Another immune checkpoint that can be targeted in the methods provided herein is the T-cell immunoglobulin and mucin-domain containing-3 (TIM-3), also known as hepatitis A virus cellular receptor 2 (HAVCR2) and CD366. The complete mRNA sequence of human TIM-3 has the Genbank accession number NM_032782. TIM-3 is found on the surface IFNγ-producing CD4⁺ Th1 and CD8⁺ Tc1 cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4⁺ Th1-specific cell surface protein that regulates macrophage activation. Inhibitors of the disclosure may block one or more functions of TIM-3 activity.

In some embodiments, the immune checkpoint inhibitor is an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or VH and/or VL domains derived therefrom) suitable for use in the present methods can be generated using methods well known in the art. Alternatively, art recognized anti-TIM-3 antibodies can be used. For example, anti-TIM-3 antibodies including: MBG453, TSR-022 (also known as Cobolimab), and LY3321367 can be used in the methods disclosed herein. These and other anti-TIM-3 antibodies useful in the claimed invention can be found in, for example: U.S. Pat. Nos. 9,605,070, 8,841,418, US2015/0218274, and US 2016/0200815. The teachings of each of the aforementioned publications are hereby incorporated by reference. Antibodies that compete with any of these art-recognized antibodies for binding to LAG3 also can be used.

In some embodiments, the inhibitor comprises the heavy and light chain CDRs or VRs of an anti-TIM-3 antibody. Accordingly, in one embodiment, the inhibitor comprises the CDR1, CDR2, and CDR3 domains of the VH region of an anti-TIM-3 antibody, and the CDR1, CDR2 and CDR3 domains of the VL region of an anti-TIM-3 antibody. In another embodiment, the antibody has at least about 70, 75, 80, 85, 90, 95, 97, or 99% (or any derivable range therein) variable region amino acid sequence identity with the above-mentioned antibodies.

### 2. Activation of Co-Stimulatory Molecules

In some embodiments, the immunotherapy comprises an activator (i.e., agonist) of a co-stimulatory molecule. In some embodiments, the activator comprises an activator of B7—1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Activators include agonistic antibodies, polypeptides, compounds, and nucleic acids.

### 3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7. TLR8 or CD40 have been used as antibody targets.

### 4. CAR-T and CAR-NK Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell, natural killer (NK) cell, or other immune cell. The receptors are called chimeric because they are fused of parts from different sources. CAR-T cell therapy refers to a treatment that uses such transformed cells for cancer therapy, where the transformed cells are T cells. Similar therapies include, for example, CAR-NK cell therapy, which uses transformed NK cells.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Example CAR-T therapies include Tisagenlecleucel (Kymriah) and Axicabtagene ciloleucel (Yescarta).

### 5. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an example interleukin cytokine therapy.

### 6. Adoptive T-Cell Therapy

Adoptive T cell therapy (ACT) is a form of passive immunization by the transfusion of T-cells (adoptive cell transfer). They are found in blood and tissue and usually activate when they find foreign pathogens. In particular, they may activate when a T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, which may limit or prevent immune-mediated tumor death.

Multiple ways of producing and obtaining tumor targeted T cells have been developed. T-cells specific to a tumor antigen can be removed from a tumor sample (tumor-infiltrating lymphocytes, or “TILs”) or filtered from blood. Subsequent activation and culturing may be performed ex vivo, with the resulting cells administered to a subject. Activation can take place through gene therapy and/or or by exposing the T cells to tumor antigens. T cells can be genetically modified to alter one or more properties, for example enhancement of therapeutic efficacy. In some embodiments. T cells are genetically modified prior to being administered to a subject. In some embodiments, T cells that do not secrete TGF-β1 are used for adoptive T-cell therapy. In some embodiments. T cells that do not express TGF-β1 (e.g., T cells comprising a knockout mutation of TGFB1) are used for adoptive T-cell therapy. Genetically modified T cells, and methods for generating such cells, are discussed further elsewhere herein.

### C. Oncolytic Virus

In some embodiments, the additional therapy comprises an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy

### D. Polysaccharides

In some embodiments, the additional therapy comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

### E. Neoantigens

In some embodiments, the additional therapy comprises neoantigen administration. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

### F. Chemotherapies

In some embodiments, the additional therapy comprises a chemotherapy. Suitable classes of chemotherapeutic agents include (a) Alkylating Agents, such as nitrogen mustards (e.g., mechlorethamine, cyclophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozotocin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophyllotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydrazine derivatives (e.g., procarbazine), and adrenocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m² to about 20 mg/m² for 5 days every three weeks for a total of three courses being contemplated in certain embodiments. In some embodiments, the amount of cisplatin delivered to the cell and/or subject in conjunction with the construct comprising an Egr-1 promoter operatively linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m². Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### G. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### H. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### III. Administration of Therapeutic Compositions

A therapy provided herein may comprise administration of a combination of therapeutic agents, such as a first cancer therapy (e.g., an immunotherapeutic such as genetically modified T cells that do not express TGF-β1) and a second cancer therapy (e.g., a chemotherapeutic, an additional immunotherapeutic, etc.). The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 M; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### IV. Proteins

As used herein, a “protein” or “polypeptide” refers to a molecule comprising at least five amino acid residues. A “peptide.” as used herein, refers to a molecule comprising at least three amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

### A. TGF-β1

Aspects of the disclosure relate to transforming growth factor beta-1 (TGF-β1). TGF-β1 (NCBI Protein RefSeq NP_000651) is a cytokine encoded by the gene TGFB1 (NCBI mRNA Refseq NM_000660). Disclosed, in some embodiments, are cells having one or more genetically engineered mutations in the TGFB1 gene.

### B. TGF-β2

Aspects of the disclosure relate to transforming growth factor beta-2 (TGF-ß2). TGF-β2 (NCBI Protein RefSeq NP_001129071) is a cytokine encoded by the gene TGFB2 (NCBI mRNA Refseq NM_001135599). Disclosed, in some embodiments, are cells having one or more genetically engineered mutations in the TGFB2 gene.

### C. TGF-β3

Aspects of the disclosure relate to transforming growth factor beta-1 (TGF-β3). TGF-β3 (NCBI Protein RefSeq NP_003230) is a cytokine encoded by the gene TGFB3 (NCBI mRNA Refseq NM_003239). Disclosed, in some embodiments, are cells having one or more genetically engineered mutations in the TGFB3 gene.

### V. Cells and Formulations

Certain embodiments relate to cells comprising polypeptides or nucleic acids of the disclosure. In some embodiments the cell is an immune cell. Immune cells contemplated herein include, but are not limited to, T cells, B cells, natural killer cells, and the like. In some embodiments, the immune cell is a T cell. “T cell” includes all types of immune cells expressing CD3 including T-helper cells, natural killer T (NKT) cells, cytotoxic T cells, T-regulatory cells (Treg), and gamma-delta T cells. The T cell may refer to a CD4+ or CD8⁺ T cell. Immune cells contemplated herein include engineered immune cells, including immune cells engineered to express one or more exogenous proteins.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), human embryonic kidney (HEK) 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3). HLHepG2 cells, Hut-78, Jurkat, HL-60, NK cell lines (e.g., NKL, NK92, and YTS), and the like.

In some instances, the cell is not an immortalized cell line, but is instead a cell (e.g., a primary cell) obtained from an individual. For example, in some cases, the cell is an immune cell obtained from an individual. As an example, the cell is a T lymphocyte obtained from an individual. As another example, the cell is a cytotoxic cell obtained from an individual. As another example, the cell is a stem cell (e.g., peripheral blood stem cell) or progenitor cell obtained from an individual.

In particular embodiments, the cells of the disclosure may be specifically formulated and/or they may be cultured in a particular medium. The cells may be formulated in such a manner as to be suitable for delivery to a recipient without deleterious effects.

The medium in certain aspects can be prepared using a medium used for culturing animal cells as their basal medium, such as any of AIM V. X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco).

In certain embodiments, the medium may comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and/or T3 (triodo-I-thyronine). In specific embodiments, one or more of these may be explicitly excluded.

In some embodiments, the medium further comprises vitamins. In some embodiments, the medium comprises 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. In some embodiments, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. In some embodiments, the vitamins include or consist essentially of biotin, DL alpha tocopherol acetate, DL alpha-tocopherol, vitamin A, or combinations or salts thereof. In some embodiments, the medium further comprises proteins. In some embodiments, the proteins comprise albumin or bovine serum albumin, a fraction of BSA, catalase, insulin, transferrin, superoxide dismutase, or combinations thereof. In some embodiments, the medium further comprises one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. In some embodiments, the medium comprises one or more of the following: a B-27® supplement, xeno-free B-27® supplement, GS21TM supplement, or combinations thereof. In some embodiments, the medium comprises or further comprises amino acids, monosaccharides, inorganic ions. In some embodiments, the amino acids comprise arginine, cystine, isoleucine, leucine, lysine, methionine, glutamine, phenylalanine, threonine, tryptophan, histidine, tyrosine, or valine, or combinations thereof. In some embodiments, the inorganic ions comprise sodium, potassium, calcium, magnesium, nitrogen, or phosphorus, or combinations or salts thereof. In some embodiments, the medium further comprises one or more of the following: molybdenum, vanadium, iron, zinc, selenium, copper, or manganese, or combinations thereof. In certain embodiments, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27@ supplement, xeno-free B-27® supplement, GS21TM supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese. In specific embodiments, one or more of these may be explicitly excluded.

The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts. In specific embodiments, one or more of these may be explicitly excluded.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein.

In specific embodiments, the cells of the disclosure are specifically formulated. They may or may not be formulated as a cell suspension. In specific cases they are formulated in a single dose form. They may be formulated for systemic or local administration. In some cases the cells are formulated for storage prior to use, and the cell formulation may comprise one or more cryopreservation agents, such as DMSO (for example, in 5% DMSO). The cell formulation may comprise albumin, including human albumin, with a specific formulation comprising 2.5% human albumin. The cells may be formulated specifically for intravenous administration; for example, they are formulated for intravenous administration over less than one hour. In particular embodiments the cells are in a formulated cell suspension that is stable at room temperature for 1, 2, 3, or 4 hours or more from time of thawing.

In particular embodiments, the cells of the disclosure comprise an exogenous T cell receptor (TCR), which may be of a defined antigen specificity. In some embodiments, a TCR is an engineered TCR. In some embodiments, the TCR can be selected based on absent or reduced alloreactivity to the intended recipient (examples include certain virus-specific TCRs, xeno-specific TCRs, or cancer-testis antigen-specific TCRs). In the example where the exogenous TCR is non-alloreactive, during T cell differentiation the exogenous TCR suppresses rearrangement and/or expression of endogenous TCR loci through a developmental process called allelic exclusion, resulting in T cells that express only the non-alloreactive exogenous TCR and are thus non-alloreactive. In some embodiments, the choice of exogenous TCR may not necessarily be defined based on lack of alloreactivity. In some embodiments, the endogenous TCR genes have been modified by genome editing so that they do not express a protein. Methods of gene editing such as methods using the CRISPR/Cas9 system are known in the art and described herein.

In some embodiments, the cells of the disclosure comprise one or more chimeric antigen receptors (CARs). Examples of tumor cell antigens to which a CAR may be directed include at least 5T4, 8H9, avB6 integrin, BCMA, B7-H3, B7-H6, CAIX, CA9, CD19, CD20, CD22, CD30, CD33, CD38, CD44, CD44v6, CD44v7/8, CD70, CD123, CD138, CD171, CEA, CSPG4, EGFR, EGFR family including ErbB2 (HER2), EGFRVIII, EGP2, EGP40, ERBB3, ERBB4, ErbB3/4, EPCAM, EphA2, EpCAM, folate receptor-a, FAP, FBP, fetal AchR, FRa, GD2, G250/CAIX, GD3, Glypican-3 (GPC3), Her2, IL-13Rx2, Lambda, Lewis-Y, Kappa, KDR, MAGE, MCSP, Mesothelin, Muc1, Muc16, NCAM, NKG2D Ligands, NY-ESO-1, PRAME, PSC1, PSCA, PSMA, RORI, SP17, Survivin, TAG72, TEMs, carcinoembryonic antigen, HMW-MAA, AFP, CA-125, ETA, Tyrosinase, MAGE, laminin receptor, HPV E6, E7, BING-4, Calcium-activated chloride channel 2, Cyclin-B1, 9D7, EphA3, Telomerase, SAP-1, BAGE family, CAGE family, GAGE family, MAGE family, SAGE family, XAGE family, NY-ESO-1/LAGE-1, PAME, SSX-2, Melan-A/MART-1, GP100/pmel17, TRP-1/-2, P. polypeptide, MC1R, Prostate-specific antigen, β-catenin, BRCA1/2, CML66, Fibronectin, MART-2, TGF-βRII, or VEGF receptors (e.g., VEGFR2), for example. The CAR may be a first, second, third, or more generation CAR. The CAR may be bispecific for any two nonidentical antigens, or it may be specific for more than two nonidentical antigens.

### VI. Kits

Certain aspects of the present disclosure also concern kits containing compositions of the invention or compositions to implement methods of the invention. In some embodiments, kits can be used to evaluate one or more biomarkers. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, there are kits for evaluating biomarker activity in a cell.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20×or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, probes, and inhibitors are included in some kit embodiments. In addition, a kit may include a sample that is a negative or positive control for methylation of one or more biomarkers.

Any embodiment of the disclosure involving specific biomarker by name is contemplated also to cover embodiments involving biomarkers whose sequences are at least 80, 81, 82, 83, 84, 85, 86, 87, 88, 89, 90, 91, 92, 93, 94, 95, 96, 97, 98, 99% identical to the mature sequence of the specified nucleic acid.

Embodiments of the disclosure include kits for analysis of a pathological sample by assessing biomarker profile for a sample comprising, in suitable container means, two or more biomarker probes, wherein the biomarker probes detect one or more of the biomarkers identified herein. The kit can further comprise reagents for labeling nucleic acids in the sample. The kit may also include labeling reagents, including at least one of amine-modified nucleotide, poly(A) polymerase, and poly(A) polymerase buffer. Labeling reagents can include an amine-reactive dye.

It is contemplated that any method or composition described herein can be implemented with respect to any other method or composition described herein and that different embodiments may be combined. The claims originally filed are contemplated to cover claims that are multiply dependent on any filed claim or combination of filed claims.

### Examples

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Example 1—Depletion of Endogenous TGF-β1 in CD8⁺ T Cells Via Knockout of TGFB1

### Results

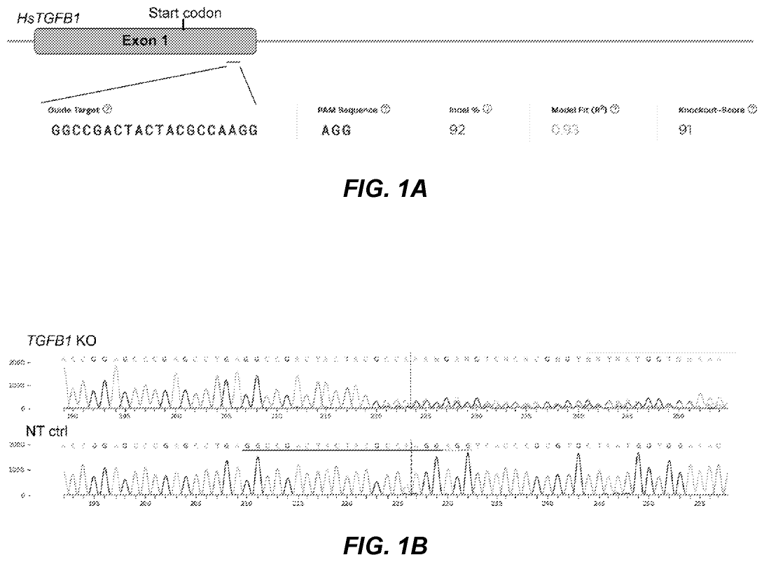

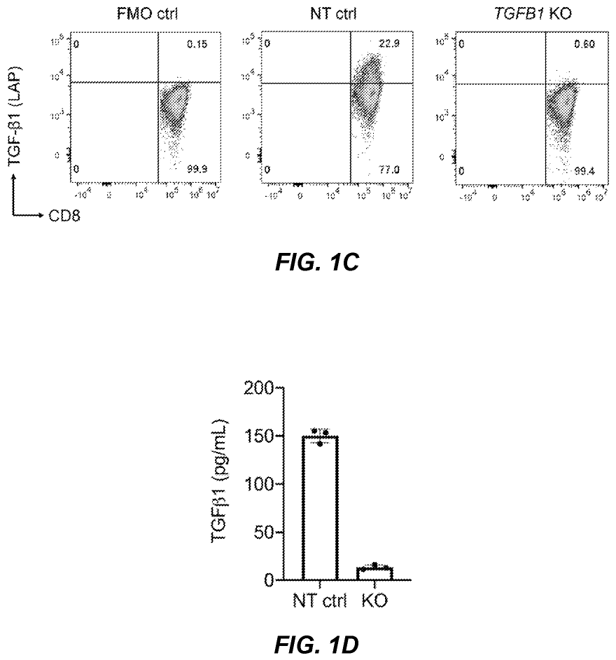

In order to deplete the TGF-β1-encoding gene, TGFB1, in CD8⁺ T cells, CRISPR-Cas9 gene editing technology was used. The efficiency of multiple gRNAs were tested in a-CD3/α-CD28 beads-stimulated naïve T cells and the gRNA with the highest knockout efficiency was selected. The selected gRNA had a guide target sequence of GGCCGACUACUACGCCAAGG (SEQ ID NO:1). This gRNA targeted the 3′ region of the 1^{st} exon of TGFB1 gene, downstream of the start codon (FIG. 1A). The comparison of Sanger sequencing results of non-target (NT) control gRNA-transfected T cells and those of TGFB1 gRNA-transfected ones showed that the TGFB1 DNA sequence was efficiently disrupted by this gRNA with a high insertion/deletion percentage and knockout score (FIGS. 1A and 1B). At Day 2 after beads stimulation, the membrane-bound TGF-β1 level was greatly reduced in TGFB1 knockout CD8+ cells, compared with NT control (FIG. 1C). Similarly, ELISA data showed that the total secreted TGF-β1 level also largely decreased by TGFB1 knockout (FIG. 1D). All these data revealed that the TGFB1 gRNA efficiently depleted TGFB1 in CD8⁺ T cells at both DNA and protein level.

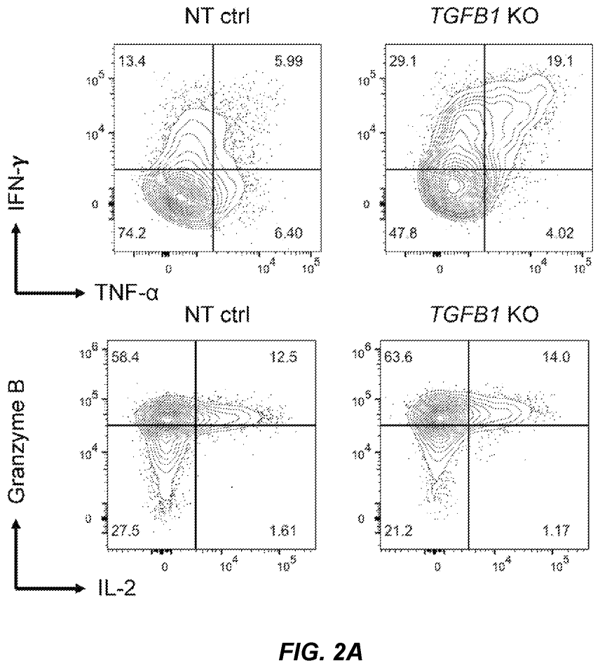

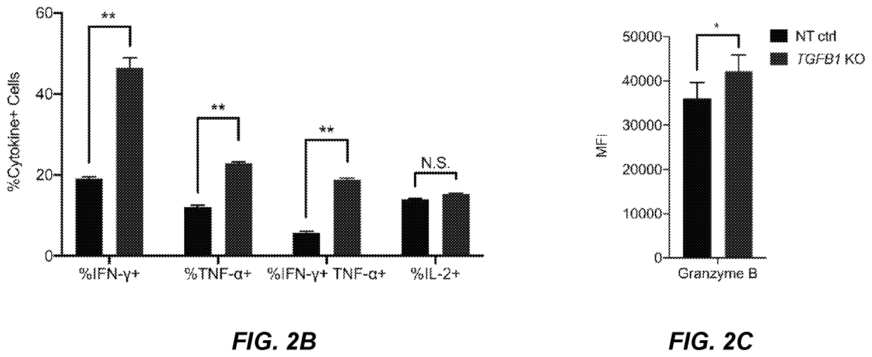

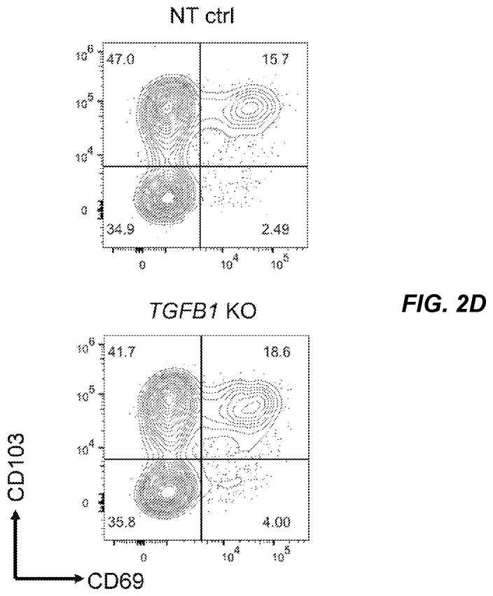

To determine whether TGFB1 knockout affected the effector functions of poly-clonally activated CD8⁺ T cells, intracellular cytokine staining was performed in control and knockout CD8⁺ T cells after re-stimulation. The TGFB1 knockout T cells produced much more TNF-α and IFN-γ (FIGS. 2A and 2B), and slightly more Granzyme B (FIGS. 2A and 2C), compared with NT control, while IL-2 level was not dramatically affected (FIGS. 2A and 2B). These results suggested that TGFB1 knockout in CD8⁺ T cells enhanced cytokine production. Next, the capability to form resident memory T cells (T_{RM}) was compared between NT control and knockout cells. In response to hypoxia and exogenous TGF-β1, conditions sufficient to induce T_{RM} formation, the percentage of CD103+CD69+T_{RM} population was roughly similar between control and TGFB1 knockout T cells, indicating that the depletion of endogenous TGF-β1 from CD8⁺ T cells did not dramatically affect T_{RM} formation.

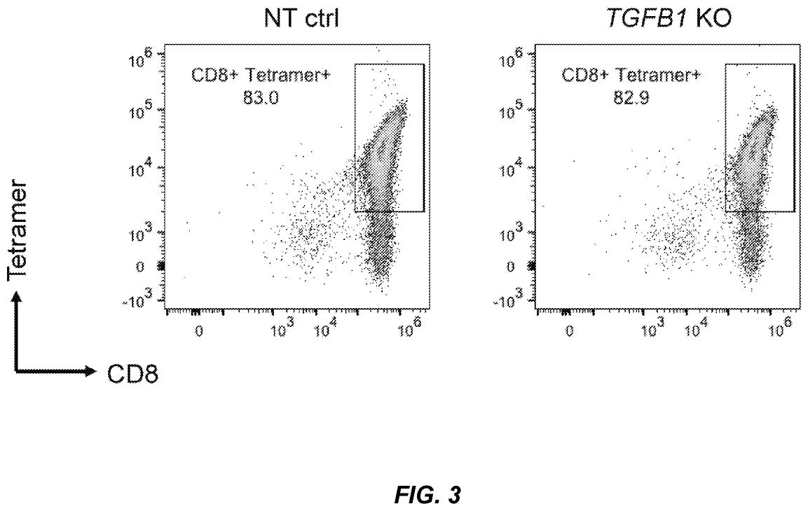

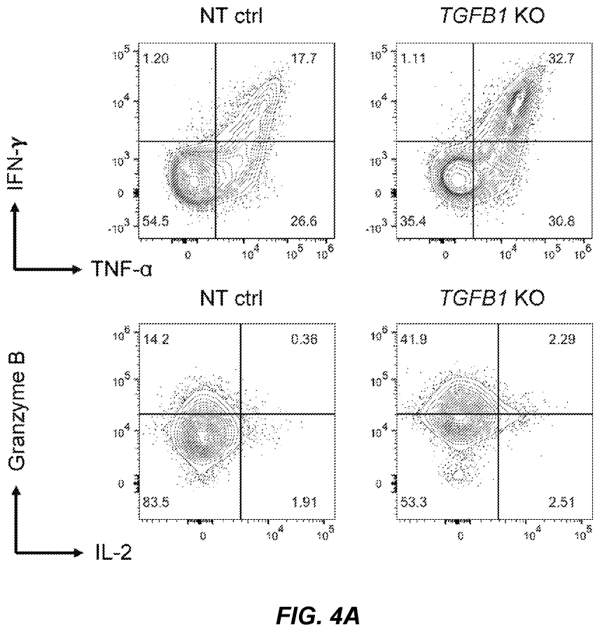

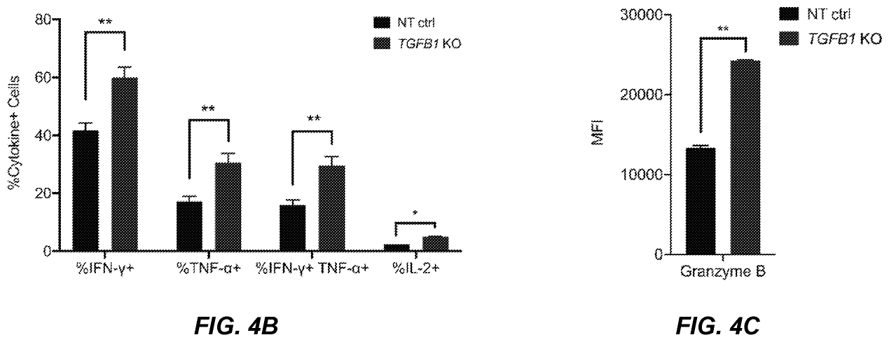

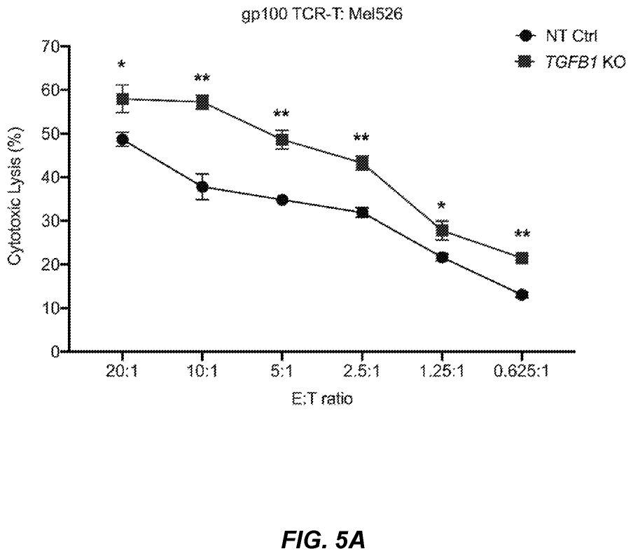

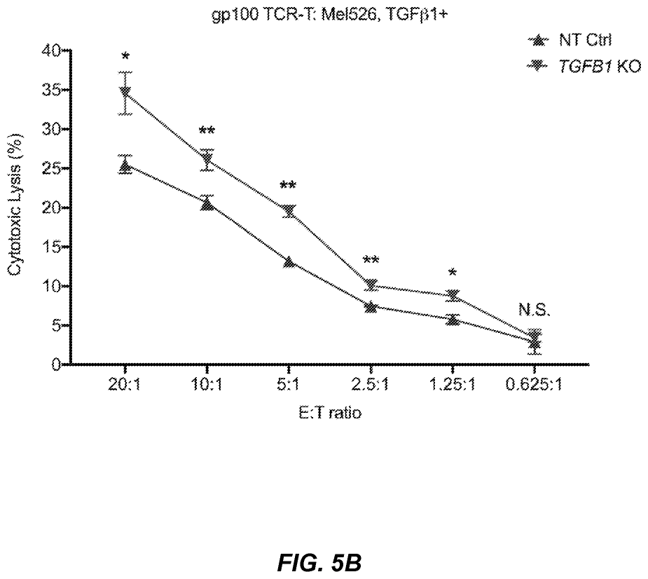

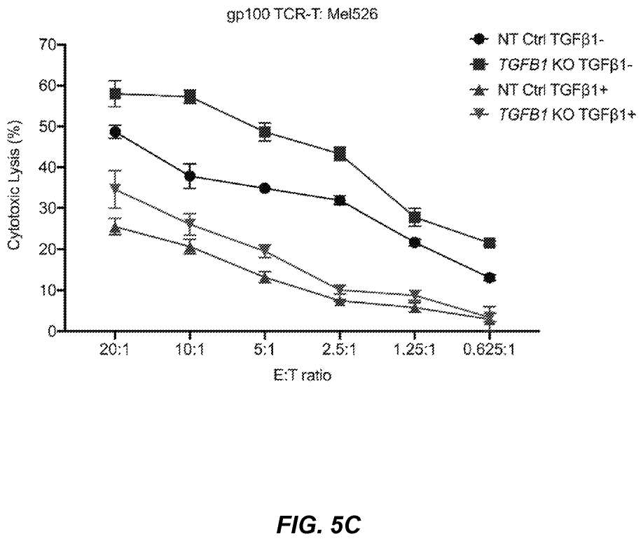

In order to determine the effect of TGFB1 knockout in an antigen-specific setting, the T cells were engineered to stably express gp100-specific, HLA-A*0201-restricted TCR (TCR-T). Knockout was performed in sorted tetramer⁺ CD8⁺ TCR-T cells and cells were then expanded using the rapid expansion protocol (REP) (FIG. 3). Intracellular cytokine staining was performed in control and TGFB1 knockout TCR-T cells after co-cultured with gp100+ tumor cell line Mel526 and it was found that TGFB1 knockout TCR-T cells produced much more IFN-γ. TNF-α and Granzyme B than control cells, which was consistent with the results from the poly-clonally stimulated CD8+ T cells (FIGS. 4A-4C). In addition, the IL-2 level was also slightly higher in knockout TCR-T cells than in NT control (FIGS. 4A and 4B). The cytotoxicity of NT control and TGFB1 knockout TCR-T cells co-cultured with Mel526 cells was measured by chromium release assay. TGFB1 knockout TCR-T cells showed increased cytolytic activity to Mel526 cells at various E:T ratios compared with control cells (FIG. 5A). In order to compare the cytolytic activity of control and TGFB1 knockout TCR-T cells in the high-exogenous-TGF-β1 condition, TCR-T cells were pretreated with high-dose TGF-β1 for 4 days before chromium release assay. Interestingly, even in the presence of high-dose exogenous TGF-β1, TGFB1 knockout TCR-T cells were still more efficient than NT control in killing target cells (FIG. 5B). In addition, both control and TGFB1 knockout TCR-T cells in the presence of exogenous TGF-β1 showed decreased cytolytic activity, compared with the same cells without exogenous TGF-β1 (FIG. 5C). In summary, TGFB1 knockout dramatically enhanced the anti-tumor effector functions of CD8+ T cells, which indicates a great potential to improve efficacy of adoptive T cell therapy (ACT) for treatment of cancer patients.

### Methods

### Cells and Reagents

Healthy donor peripheral blood mononuclear cells (PBMCs) were collected by leukapheresis and stored in liquid nitrogen until use. All human sample collection was performed with informed consent and approved by the institutional review board (IRB) of UT MD Anderson Cancer Center. Recombinant Cas9 enzyme and human TGFB1 guide RNA was purchased from Synthego. All flow cytometry antibodies and human TGF-β1 ELISA kit were purchased from Biolegend. MHC-I-gp100 tetramer was from Fred Hutchinson Cancer Research Center.

### Polyclonal Stimulation of CD8⁺ T Cells

Naïve CD8⁺ T cells (CD8⁺ CD45RA⁺ CCR7⁺) were flow cytometry-sorted and the purity of the naïve CD8+ T cells was greater than 99% as determined by flow cytometry. CD8⁺ T cells were activated using Dynabeads® Human T-Activator CD3/CD28 for T-Cell Expansion and Activation (Life Technologies) at a bead:cell ratio of 1:4.

### In Vitro Induction of Resident Memory T Cells

Sorted naïve CD8⁺ T cells (CD8⁺ CD45RA⁺ CCR7⁺) were activated using Dynabeads® Human T-Activator CD3/CD28 for T-Cell Expansion and Activation (Life Technologies) at a bead:cell ratio of 1:4 in 2% 02. After 4 days, 1.25 ng/mL recombinant TGF-β1 was added. After another 2 days, T cells were harvested and beads were removed using a magnet before downstream analysis.

### Retroviral Infection

Retroviral supernatant was produced in the 293GP packaging cell line. In brief, In brief, 70% confluent 293GP 10-cm plates were co-transfected with 10 μg MSGV vector plasmid and 5 μg RD114 envelope plasmid DNA using Lipofectamine 3000. Medium was replaced at Day 1 after transfection. On Day 3, viral supernatants were collected, centrifuged to remove cell debris, and frozen at −80° C. for future use. Healthy donor PBMC (HLA-A* 0201) were thawed and activated same day with 30 ng/mL anti-CD3 (OKT3) and 300 U/ml IL-2. After 3 days, activated T cells were transduced with retroviral supernatants by 2 h centrifuge at 32° ° C. and 2,000 g, and then cultured for 3 more days before sorting for tetramer-positive CD8⁺ T cells.

### CRISPR-Cas9 Knockout

CRISPR-Cas9 gene knockout was performed by transient Cas9/gRNA ribonucleoprotein (RNP) complex electroporation using the P3 Primary Cell 4D-Nucleofector X Kit S (Lonza). Sorted TCR-T or activated naïve CD8+ T cells were washed and resuspended in P3 buffer at 1×10⁶ cells per 20 μl reaction. 40 pmol recombinant SpCas9 protein and 80 pmol chemically modified synthetic sgRNA (2:1 molar ratio gRNA:Cas9) per reaction was pre-complexed for 15 min at room temperature to create RNP complexes. A 20-μl cell suspension was mixed with RNP and electroporated using the EH-115 protocol in 16-well cuvette strips. Cells were recovered at 37 ºC for 15 min then cultured in 300 U/ml IL-2.

### Cell Culture and Rapid Expansion Protocol (REP)

The medium for CD8+ T cells was RPMI1640, 10% FBS, 4 mM Glutamine, and 2-Mercaptoethanol. For REP, CTL lines were expanded using 30 ng/mL anti-CD3 (OKT3) and 200×irradiated allogeneic PBMCs or LCLs as feeder cells. The cultures were fed with IL-2 at 50 U/ml every 3 days. After 14 days, expanded cells were subjected to further analyses. Mel526 cells were cultured in RPMI1640, 10% FBS, 4 mM Glutamine, 1× Non-essential Amino Acids, 1 mM Sodium Pyruvate, and 1% penicillin/streptomycin. All cells were tested for mycoplasma using a PCR-based test and used within one month after thawing cryopreserved stock vials.

### Flow Cytometry

At specific time points during culture, cells were stained with antibodies against CD8, LAP, CD103, CD69, IL-2, IFN-γ. TNF-α or Granzyme B or MHC-I-gp100 peptide tetramer. For intracellular cytokine staining, cells were re-stimulated with anti-CD3/CD28 beads in the presence of Brefeldin A for 16 hours, fixed and permeabilized before staining. All FACS data were acquired via a Novocyte flow cytometer and analyzed via FlowJo software (Tree Star, Inc.).

### Chromium Release Assay

Tumor target cells were labeled with 100 μCi 51Cr for 2 hours. After washing, labeled tumor target cells were plated at 2,000 cells/well of 96-well V-bottom plates (4 repeats) and incubated with 20,000 TCR-T cells at various effector: tumor (E:T) ratio for 4 hours. The negative control was labeled tumor target cells without effector T cells, and the positive control was tumor target cells incubated with Trypan lysis buffer (0.4% Trypan blue, 10% Nonidet P40). Then 30 μl supernatants from each well were collected and the 51Cr amount in the supernatants was measured with MicroBeta Microplate counter (PerkinElmer) and the killing efficiency was calculated as % killing=100%×(sample average−average of negative control)/(average of positive control−average of negative control).

### Statistical Analysis

Graphical presentation and statistical analysis of the data were performed using GraphPad Prism (Version 7, GraphPad software, San Diego, CA) and Excel. Data are displayed as mean and STD. Results between experimental groups were compared using Student's t test. p<0.05 was considered statistically significant. Statistical significance is displayed as * p<0.05. ** p<0.01.

All of the methods disclosed and claimed herein can be made and executed without undue experimentation in light of the present disclosure. While the compositions and methods of this invention have been described in terms of certain embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed:

1. A method for treating a subject having cancer, the method comprising administering to the subject a human CD8⁺ T cell that does not secrete TGF-β1.

2. The method of claim 1, wherein the human CD8⁺ T cell does not express TGF-β1.

3. The method of claim 1, wherein the human CD8⁺ T cell expresses a mutant TGF-β1 protein.

4. The method of claim 1, wherein the human CD8⁺ T cell was derived from a CD8⁺ T cell from the subject.

5. The method of claim 4, wherein the human CD8⁺ T cell from the subject is a tumor-infiltrating lymphocyte.

6. The method of any of claims 1-5, wherein the human CD8⁺ T cell comprises a mutation of TGFB1.

7. The method of claim 6, where the mutation is a frameshift mutation.

8. The method of claim 6, wherein the mutation is a nonsense mutation.

9. The method of claim 6, wherein the mutation is a modification of a region of exon 1 of TGFB1.

10. The method of claim 9, wherein the region of exon 1 comprises the sequence of SEQ ID NO:1.

11. The method of any of claims 1-10, wherein the cancer is melanoma.

12. The method of any of claims 1-11, wherein the cancer is recurrent cancer.

13. The method of any of claims 1-12, wherein the subject was previously treated for cancer with a previous treatment.

14. The method of claim 13, wherein the subject was determined to be resistant to the previous treatment.

15. The method of any of claims 1-14, wherein the method comprises administering to the subject a population of human CD8⁺ T cells comprising the human CD8⁺ T cell, wherein each cell of the population of human CD8⁺ T cells does not secrete TGF-β1.

16. The method of any of claims 1-15, further comprising administering an additional cancer therapy.

17. The method of claim 16, wherein the additional cancer therapy is a chemotherapy, radiotherapy, or immunotherapy.

18. A method for genetically modifying a human CD8⁺ T cell, the method comprising introducing into the cell a Cas nuclease and a guide RNA targeted to TGFB1.

19. The method of claim 18, wherein the guide RNA is targeted to a region of exon 1 of TGFB1.

20. The method of claim 19, wherein the region of exon 1 comprises the sequence of SEQ ID NO:1.

21. The method of any of claims 18-20, wherein the Cas nuclease is Cas9.

22. The method of any of claims 18-20, wherein the Cas nuclease is Cas12a.

23. The method of any of claims 18-22, wherein a mutation is generated in TGFB1 in the cell.

24. The method of claim 23, wherein the mutation is generated using the Cas nuclease.

25. The method of claim 23 or 24, where the mutation is a frameshift mutation.

26. The method of claim 23 or 24, wherein the mutation is a nonsense mutation.

27. The method of any of claims 18-26, wherein the Cas nuclease and the guide RNA are introduced into the cell via transfection.

28. The method of any of claims 18-26, wherein the Cas nuclease and the guide RNA are introduced into the cell via electroporation.

29. The method of any of claims 18-28, further comprising obtaining the human CD8⁺ T cell from a subject.

30. The method of any of claims 18-29, wherein the human CD8⁺ T cell is a tumor-infiltrating lymphocyte.

31. A method for treating a subject having cancer, the method comprising:
(a) modifying immune cells from the subject to generate genetically modified immune cells that do not secrete TGF-β1; and
(b) administering the genetically modified immune cells to the subject.

32. The method of claim 31, wherein the genetically modified immune cells do not express TGF-β1.

33. The method of claim 31, wherein the genetically modified immune cells express a mutant TGF-β1 protein.

34. The method of claim 31, wherein secretion of TGF-β1 is undetectable in the genetically modified immune cells.

35. The method of claim 31, wherein secretion of TGF-β1 is decreased by at least 80% in the genetically modified immune cells compared to a population of control or reference cells.

36. The method of claim 31, wherein secretion of TGF-β1 is decreased by at least 90% in the genetically modified immune cells compared to a population of control or reference cells.

37. The method of claim 31, wherein expression of TGF-β1 is undetectable in the genetically modified immune cells.

38. The method of claim 31, wherein expression of TGF-β1 is decreased by at least 80% in the genetically modified immune cells compared to a population of control or reference cells.

39. The method of claim 31, wherein expression of TGF-β1 is decreased by at least 90% in the genetically modified immune cells compared to a population of control or reference cells.

40. The method of any of claims 31-39, wherein the immune cells are natural killer T cells.

41. The method of any of claims 31-39, wherein the immune cells are modified to express a cloned T cell receptor (TCR) or a chimeric antigen receptor (CAR).

42. The method of any of claims 31-39, wherein the immune cells are engineered CAR-T cells or engineered TCR-T cells.

43. The method of any of claims 31-39, wherein the immune cells are T cells.

44. The method of claim 43, wherein the T cells are CD4⁺ T cells.

45. The method of claim 43, wherein the T cells are CD8⁺ T cells.

46. The method of any of claims 31-45, wherein the immune cells are tumor-infiltrating lymphocytes.

47. The method of any of claims 31-46, wherein modifying the immune cells comprises generating a mutation in TGFB1.

48. The method of claim 47, where the mutation is a frameshift mutation.

49. The method of claim 47, wherein the mutation is a nonsense mutation.

50. The method of any of claims 47-49, wherein the mutation is a mutation in exon 1 of TGFB1.

51. The method of any of claims 31-50, wherein modifying the immune cells comprises targeting a region of exon 1 of TGFB1 using a Cas nuclease and a guide RNA.

52. The method of claim 51, wherein the region of exon 1 comprises SEQ ID NO:1.

53. The method of any of claims 31-52, wherein the subject is a human subject.

54. The method of any of claims 31-53, further comprising, prior to (a), obtaining the immune cells from the subject.

55. The method of any of claims 31-54, wherein the cancer is melanoma.

56. The method of any of claims 31-55, wherein the cancer is recurrent cancer.

57. The method of any of claims 31-56, wherein the subject was previously treated for cancer with a previous treatment.

58. The method of claim 57, wherein the subject was determined to be resistant to the previous treatment.

59. The method of any of claims 31-58, further comprising administering an additional cancer therapy.

60. The method of claim 59, wherein the additional cancer therapy is a chemotherapy, radiotherapy, or immunotherapy.

61. A human immune cell comprising a genetic modification that prevents the cell from secreting TGF-β1.

62. The cell of claim 61, wherein the immune cell is a T cell.

63. The cell of claim 62, wherein the T cell is a CD8⁺ T cell.

64. The cell of claim 62, wherein the T cell is a CD4⁺ T cell.

65. The cell of claim 61, wherein the immune cell is a natural killer T cell.

66. The cell of claim 61, wherein the immune cell expresses a cloned TCR or a CAR.

67. The cell of claim 61, wherein the immune cell is an engineered CAR-T cell or an engineered TCR-T cell.

68. The cell of any of claims 61-67, wherein the genetic modification is a frameshift mutation of TGFB1.

69. The cell of any of claims 61-67, wherein the genetic modification is a nonsense mutation of TGFB1.

70. The cell of any of claims 61-67, wherein the genetic modification is a deletion of TGFB1.

71. The cell of any of claims 61-67, wherein the genetic modification comprises a modification of a region of exon 1 of TGFB1.

72. The cell of claim 71, wherein the region of exon 1 comprises SEQ ID NO:1.

73. The cell of any of claims 61-72, wherein the cell is or was derived from a tumor-infiltrating lymphocyte.

74. A population of human immune cells comprising the human immune cell of any of claims 61-73.

75. The population of human immune cells of claim 74, wherein secretion of TGF-β1 is undetectable in the population of human immune cells.

76. The population of human immune cells of claim 74, wherein secretion of TGF-β1 is decreased by at least 80% in the population of human immune cells compared to a population of control or reference cells.

77. The population of human immune cells of claim 74, wherein secretion of TGF-β1 is decreased by at least 90% in the population of human immune cells compared to a population of control or reference cells.

78. The population of human immune cells of any of claims 74-77, wherein expression of TGF-β1 is undetectable in the population of human immune cells.

79. The population of human immune cells of any of claims 74-77, wherein expression of TGF-β1 is decreased by at least 80% in the population of human immune cells compared to a population of control or reference cells.

80. The population of human immune cells of any of claims 74-77, wherein expression of TGF-β1 is decreased by at least 90% in the population of human immune cells compared to a population of control or reference cells.

81. A pharmaceutical composition comprising (a) the human immune cell of any of claims 61-73 or the population of human immune cells of any of claims 74-80 and (b) a pharmaceutically acceptable excipient.

82. A method for treating a subject having cancer comprising administering to the subject the human immune cell of any of claims 61-73, the population of human immune cells of any of claims 74-80, or the pharmaceutical composition of claim 81.

83. The method of claim 82, wherein the cancer is melanoma.

84. The method of claim 82 or 83, wherein the cancer is recurrent cancer.

85. The method of any of claims 82-84, wherein the subject was previously treated for cancer with a previous treatment.

86. The method of any of claims 82-85, wherein the subject was determined to be resistant to the previous treatment.

87. The method of any of claims 82-86, wherein the human immune cell was derived from a cell from the subject.
